(19) United States
(12) Patent Application Publication
(10) Pub. No.: US 2011/0028850 A1
(43) Pub. Date: Feb. 3, 2011

(54) Process for quantitative display of blood flow

(76) Inventors: Thomas Schuhrke, Munich (DE); Guenter Meckes, Munich (DE); Joachim Steffen, Westhausen (DE); Hans-Joachim Miesner, Aalen (DE); Frank Rudolph, Aalen (DE); Werner Nahm, Buehlerzell (DE)

Correspondence Address: ECKERT SEAMANS CHERIN & MELLOTT, LLC, U.S. STEEL TOWER, 600 GRANT STREET, PITTSBURGH, PA, 15219-2788, US

(21) Appl. No.: 12/462,038

(22) Filed: Jul. 28, 2009

(51) Int. Cl. A61B 6/00; A61B 5/02

(52) U.S. Cl. 600/476; 600/504

(57) ABSTRACT

A method for the quantitative representation of the blood flow in a tissue or vascular region is based on the signal of a contrast agent injected into the blood. In the method, several individual images of the signal emitted by the tissue or vascular region are recorded at successive points in time and are stored. Based on the respective signal, a quantity characteristic for the blood flow and a quantity characteristic for the position of the blood vessels are determined for image areas of individual images. These quantities are represented superimposed for the respective image areas such that both the blood flow quantity and the position of the fine blood vessels become clearly visible in the representation and can be differentiated from the tissue.

### BACKGROUND OF THE INVENTION

The invention relates to a quantitative method for the representation (display) of the blood flow in a patient.

Several methods for observing and determining the blood flow in tissue and vascular regions are known in which a chromophore such as indocyaninin green, for example, is applied. The fluorescent dye can be observed as it spreads in the tissue or along the blood vessels using a video camera. Depending on the area of application, the observation can be non-invasive or in the course of surgery, via the camera of a surgical microscope.

Many methods are known, where only the relative distribution of the fluorescent dye in the tissue or in the blood vessels is examined qualitatively in order to draw conclusions concerning their blood flow. For example, conclusions were drawn about the blood flow and diagnoses provided by watching an IR video recorded during surgery. It is also known to record the rise in brightness of the fluorescence signal over time at all or at selected image points and in this manner record a time chart of the signal emitted by the fluorescent dye. The profile of the recorded formation plot provides the physician with information about potential vascular constrictions or other problems in the area of this image point. One example for this is provided in DE 101 20 980 A1. However, the method described in the DE 101 20 980 A1 goes beyond the qualitative analysis and embarks on a path towards a quantitative determination of the blood flow at every image point.

### SUMMARY OF THE INVENTION

The objective forming the basis of the invention is to provide medical professionals with additional aids from which they can draw conclusions concerning blood flow problems and that can support making a diagnosis.

This objective, as well as other objectives which will become apparent from the discussion that follows, are achieved, according to the present invention, by the method and apparatus described below.

According to the invention, the contrast agent flowing into the tissue or vascular area is observed by recording the signal emitted by said contrast agent as a video, by splitting the video into individual images and storing the same, or by storing individual images directly, and by determining a quantity that is derived from the respective signal for several corresponding image areas, in particular image points of the individual images, said quantity being characteristic for the blood flow, in order to generate a two-dimensional representation based on the quantity determined for the image areas. In order to ensure a better orientation and assignment of the vessels in the representation, an additional quantity is derived at the same time for each image area, with said quantity being characteristic for the position of the vessel, and being superimposed in the representation onto the determined quantity that is characteristic for the blood flow. The quantities can be determined in succession or at the same time. They can be stored individually and then superimposed or they can be superimposed directly and stored as a result for each image area. If the determined quantities can be used individually for diagnosis purposes, it is advantageous to store them individually and to superimpose them thereafter. In an ideal case, the respective image areas of the individual images can be the same local image point or image area, that is, a number of adjacent image points, if different individual images have been taken with the same resolution of exactly the same detail of the object, or according to the invention in one advantageous embodiment can also be image points or image areas in different individual images that are assigned to each other because the recording conditions have changed between the recordings, for example, object and shooting direction have moved in relation to each other or the resolution has been changed or the like. This will be explained in greater detail in a later section. The image area of several individual images is the outcome of assigning one respective image area of each individual image to the respective image area of the following individual image. A quantity or value is determined from the values that are obtained from the respective corresponding image areas of the individual images, with said quantity or value being characteristic for the blood flow in this image area as well as a quantity or a value that is characteristic for the position of the blood vessels in this image area. The obtained quantities are superimposed for this image area and are represented as superimposed quantities within the image area. Preferably, the injected contrast agent is a fluorescent dye, such as indocyaninin green, for example. However, other dyes known for perfusion diagnostics can be used as well. The excitation of the fluorescence for generating the signal to be obtained occurs typically via a near infrared light source. An infrared camera, which is often a CCD camera or a CMOS camera and which can be an autonomous medical device or can be integrated in a surgical microscope, is used for recording. The generation of the individual images of the signals that are to be analyzed occurs either by splitting a continuous video into individual images or directly through storing recorded individual images in certain time sequences, which may be stored as a bitmap, for example. The superimposed representation provides a valuable aid to the treating person in order to recognize flow blockages or constrictions and also to assign them reliably to individual vessels. It is, therefore a very important new diagnosis aid. Of course, more than two signals can be superimposed in the context of the invention as well. However, it would be advantageous to use a binary signal as a third signal or as the third quantity after the evaluation of the signal.

Preferably, the quantity characteristic for the blood flow and the quantity characteristic for the position of the blood vessels are superimposed for the image points to be represented. The value of a shown image point is a combination of a quantity over the blood flow and a quantity over the position of the blood vessels. Due to the fact that the information for both the position and the blood flow enter the image point, the individual image point presents the entire information necessary for a diagnosis such that the treating physician has all the information available and can make an evaluation.

In one exemplary embodiment, a weighted addition to the superimposition of the two quantities derived from the obtained signal is carried out. By multiplying each quantity prior to the addition by image points using a weighting factor (preferably, the weighting factors add up to one), it is possible to represent both quantities at the same time in a representation without receiving values that exceed the value range to be shown. This type of superimposition can be used particularly advantageously when a false color image and a grayscale image, for example a time offset representation and a blood vessel representation shall be represented superimposed. The inflow behavior of the blood is made transparent via the time offset display realized in false color, and the position of the blood vessels into which the blood flows is added via the grayscale contrast of the blood vessel display obtained from the totality of the individual images. The full available color range remains intact. The inflow behavior can also be represented in a differentiated manner as is the case in the time offset display alone; the blood vessels can be localized additionally. But also other quantities that describe the blood flow such as the blood flow index, for example, which shows a measure for the volume flow of the blood, can be represented advantageously for each image point in color, and can be superimposed with a preferably grayscale image that specifies the position of the blood vessels to facilitate the orientation. With this combination of a false color image for the quantity that is characteristic for the blood flow and a grayscale image for the quantity that is characteristic for the position of the blood vessels, where a color value is combined with a grayscale density value in each image point, both quantities can be represented particularly advantageously at the same time quantitatively in each image point yet can still be distinguishable separately.

In an additional advantageous embodiment, the two quantities are selected during the superimposition according to a third derived quantity that serves as a control quantity. According to the value of the control quantity, either the value of the one or the other quantity or also another value is entered at each image point of the superimposed representation. In this manner, it is advantageously possible to indicate areas in the superimposed representation that are associated with blood vessels and areas outside of these in a different manner. Suitable as quantities to be used are all derived quantities of the individual images or the individual images themselves that are suitable for superimposing. In an advantageous manner, the blood vessel position itself or an edge image of the blood vessel representation using an edge detection method can be used as the control quantity. The edge image provides a particularly good overview of the position of the blood vessels and is particularly well suited for overlaying because it offers the option of entering a fixed value at the image points that are associated with edges, i.e., where the quantity that is characteristic for the position exceeds a certain threshold value, with said fixed value displaying the position of the blood vessels, while the value for the quantity that is characteristic for the blood flow can be entered at all other image points. As an alternative, the position of the blood vessels can also be displayed via the superimposition using recordings of a color video that is often recorded at surgical microscopes parallel to the infrared video.

In an additional advantageous embodiment, the superimposition is carried out in the context of a three-dimensional representation. Preferably, the quantity characteristic for the blood flow can be represented in a two-dimensional fashion and the quantity characteristic for the position of the blood vessels can be inserted as a third dimension. The preferred representation is a perspective visualization. A three-dimensional representation may be less familiar to view and may appear more complex at a first glance; however, it definitely can integrate the entire information contents of both quantities.

In an additional preferred embodiment, the quantities are shown on different axes of a suitable color space. Advantageous in this context is an HSL color space, where hue, saturation and luminance are plotted in relation to each other. One quantity can be expressed via the hue and the other via the luminance. The third axis of the color space can be held constant or can even be used for a superimposition with a third quantity. This representation also provides a good overview image that supports the treating person in making a diagnosis because the characteristic values for the blood flow as well as their relation to the blood vessels become readily apparent.

The time offset can be viewed as a quantity that is characteristic for the blood flow and is particularly well suited for this type of superimposed representation. This is the quantity that shows when the blood flows into which area and that is advantageously determined based on exceeding a threshold value for the luminance of the fluorescence signal. Often, the blood flows at the same time into blood vessels located within close proximity or into a blood vessel and the surrounding tissue. For this reason, it is not possible to perceive these areas optically separated in an exclusive representation of the time offset. Thus, a superimposition with a quantity that expresses the position of the bloods vessels is particularly important in these cases.

An additional quantity for which the superimposition can be employed advantageously is the blood flow index. Similar blood vessels that are in close proximity to each other often exhibit a similar flow behavior. Here too it is often not possible to perceive them as separate blood vessels in a pure blood flow index representation. The resolution of the individual blood vessels and their exact position can only be recognized when the blood flow index is superimposed with a quantity that is characteristic for the blood vessel position.

In one advantageous embodiment of the invention, the time offset, that is the point in time at which the threshold value at the respective image point is exceeded, or the blood flow index is transferred into a color on a color scale such that a false color image is created based on which the flow behavior of the blood is visualized. A false color image provides a very quick and intuitive overview of the time successions. After the superimposition with a grayscale or edge image of the quantity that is characteristic for the blood vessel position, the inflow into each blood vessel can be viewed in a detailed manner.

Preferably, the false color scale is selected such that an intuitive correlation to known anatomical terms exists. For example, the arterial character is emphasized by representing early points in time, i.e., small time offsets, in red while the venous character of other areas is emphasized by representing later points in time, i.e., large time offsets, in blue. In this manner, the false color image is adjusted directly to a common manner of thinking of the treating persons, and thus provides them with a very intuitive direct overview.

In an additional preferred embodiment, the superimposed representation is done in the form of a grayscale image. Here, information is lost in the superimposed representation; however, the embodiment is suited for black-and-white reproduction. To avoid this, one signal is preferably realized as a binary value. For example, if the quantity that characterizes the blood flow is selected as a color representation that is converted to gray values, then the superimposition of the quantity that is characteristic for the blood vessel position is preferably selected as an edge image. This image provides sufficient contrast to be recognized in a grayscale image.

Preferably, the maximum achieved intensity of the signal is determined as the quantity characteristic of the position of the blood vessel for each image area, preferably for each image point, in order to generate a two-dimensional representation of the total flow based on the maximum intensities determined for the image areas, i.e., the maximum signal value achieved in all areas, a so-called blood vessel representation. Since this maximum is reached at different times at the various image areas, this representation ensures an overview of the blood flow for all regions, which is not possible by viewing the individual images. Only then is a comprehensive overview provided for all areas with a blood flow. Until now, the physician had to view the recorded video several times in order to view the blood flow in different areas of the tissue or vascular region. This made it difficult to recognize if tissue areas had a poor blood flow or none at all. Due to the blood vessel representation according to the invention, the observer is able to recognize the maximum achieved concentration of the contrast agent at the same time at every point of the tissue or vascular region by viewing one single representation. Were the superimposition to occur with only one individual image, it would only be a snapshot, and regions might be recognized by mistake as having no blood vessels because blood does not yet flow through them at the given moment, even if blood does indeed flow through them at a later point in time. This is due to the fact that in such contrast agent recordings the blood vessels are recognized as such only when blood that contains contrast agents flows through these blood vessels. At a time when no contrast agent flows through them, the recording of a blood vessel shows no contrast and thus no quantity characteristic for the position of the blood vessel. Of course, another quantity that is a measure for a strong increase in the blood flow can be used instead of the signal maximum as well, For example, the added total flow can be used or a standard deviation that is a measure for the change in the blood flow or the like can be used. It is important that it is a quantity that reflects the blood flow having reached the maximum at a point in time and may then have dropped off again. However, to use the maximum itself is a particularly simple, unambiguous and therefore preferred method. An additional advantage of using the maximum is that it can be represented preferably with a low density, i.e., very bright, and in that the superimposed quantity for the blood flow is well visible in the color representation, for example.

In one additional preferred embodiment, prior to determining the quantity that is characteristic for the blood flow or for the position of the blood vessels, a movement component is applied to the individual images preferably for all quantities that require viewing of several individual images. This means, the individual images are, if they are offset from each other, first placed on top of each such that indeed the respective associated image points can be compared when determining the points in time. The underlying problem here is that the recording unit or the object to be recorded may move during recording. In such a case, the recorded images of the signals will be, at least slightly, shifted in relation to each other, such that this shift must first be reversed if one plans to receive a steady signal progression for each image point of the recorded object. Such a steady signal progression is the prerequisite for determining in a spatially resolved manner the time when the threshold value of the signal or of any other quantity that is derived from the signal is exceed. Thus, without movement compensation, the points in time or the derived quantities could be assigned falsely to the image points and could lead to an erroneous representation of the time offset, blood flow index or other parameters. Preferably, the movement is compensated using edge detection, where edge images of the individual images are generated that can then be correlated in order to determine from it the shift vector. As soon as the shift vector of an individual image is determined, this individual image is shifted in relation to the previous image according to the shift vector. In one embodiment, the edge images of successive individual images are used for the correlation of the edge images. Preferably, however, the edge image of an individual image is correlated to a reference image that is generated by joining together the previous edge images that have already been correlated to each other. In the course of this process, this creates a reference image that includes all the edges that have occurred in the individual images that have been correlated before. Any individual image can be used as the starting reference image, or an image where the total image strength has exceeded a certain value or where it is determined in another fashion that the recorded signal has exceeded a noise level and is indeed the signal of the inflowing blood. Generating the summed up reference image is essential because individual images that are recorded at very different times can shown a totally different edge structure because the signal may have already flattened in one area when it reaches the maximum in another area. It would then not be possible to properly correlate these very different images that have been recorded at different points in time. The reference image that has been derived from all individual images with relevant data also reproduces excellently the position of the blood vessels. It is, therefore, ideally suited as a quantity for the blood vessel position and thus as a quantity for superimposition. Thus, a quantity can be used for the superimposition that occurs already at the movement compensation with no need for deriving an additional quantity.

In another advantageous embodiment, a brightness correction is applied to the individual images that takes into account changes in the recording conditions that affect the brightness of the signal. For example, the amplification factor at the camera can be adjusted such that a greater contrast range of the signal can be captured during recording. The intensity of the light source or other recording conditions can be adjusted as well such that the brightness correction may need to take several different parameters into account. For this purpose, changes in the recording conditions are stored together with the individual images, and during the brightness correction, the recorded signal values are converted to a common value range taking into account these stored data. This ensures that steady signal progression occurs at every image point.

For a full understanding of the present invention, reference should now be made to the following detailed description of the preferred embodiments of the invention as illustrated in the accompanying drawings.

### BRIEF DESCRIPTION OF THE DRAWINGS

FIG. 1 shows a schematic sequence of a method for representing the blood flow.

FIG. 2 shows an example of a sequence of a brightness plot at one image point.

FIGS. 3a and b show examples of blood vessel representations without and with movement compensation.

FIGS. 4a and b show examples of a time offset representation of false color representations converted to grayscale and as a grayscale image.

FIGS. 5a, b, and c show examples of a time offset representation, a blood vessel representation and a superimposition of these two representations.

FIG. 6 shows an example of a superimposition of a time offset representation and of an edge image of the vascular area.

FIG. 7 shows an example of a superimposition of a time offset representation and of an edge image of the vascular area in a perspective, visualized, three-dimensional representation.

FIG. 8 shows schematically a surgical microscope for carrying out the method according to the invention.

### DESCRIPTION OF THE PREFERRED EMBODIMENTS

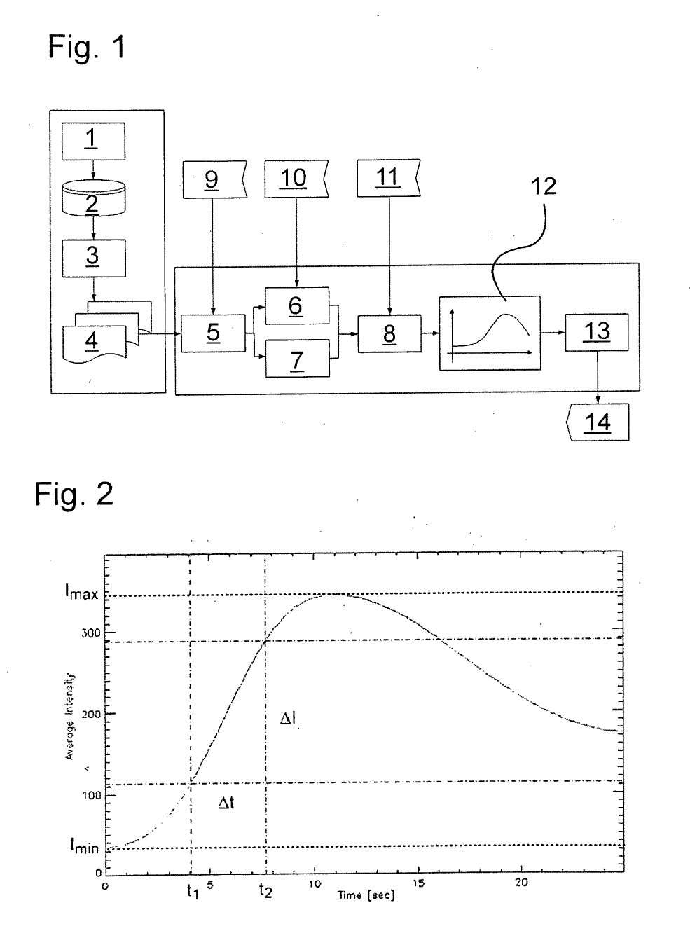

The preferred embodiments of the present invention will now be described with reference to FIGS. 1-8 of the drawings. Identical elements in the various figures are designated with the same reference numerals.

The complete system with the data flows and the individual processing steps is described in FIG. 1 and is used for representing and evaluating the blood flow. The data are recorded using a video camera 1 in the infrared range, which is arranged at the surgical microscope—not shown—or is a component thereof. The recorded infrared videos are stored in a data memory 2 and are split into individual images using a video player 3. Alternatively, it is also possible to store the images of the video camera 1 as individual images 4 from the outset. A frequency of five frames 4 per second proved to be useful. They are then corrected in a single image correction step. In the process, the corrections for the edge drop, of the dark offset or of non-linearities of the video camera 1 are carried out taking into account the required correction data 9. The data of the corrected individual images 4 are than stored in the form of compressed binary data (e.g., Motion JPEG2000 Data (MJ2)) or in the form of non-compressed binary data (e.g., bitmap). In the case of non-compressed binary data, access times are shorter and the evaluation is faster.

For the evaluation, the individual images 4 are transferred to the algorithms for the brightness correction 6 and movement correction 7. For the brightness correction 6, for example, the different amplification factors that have been set at the video camera 1 are taken into account during recording in order to adapt the video camera 1 to the different fluorescence strength of the tissue or vascular area to be recorded. They are documented during the recording as well, are stored in the data memory 2 as metadata assigned to the video data and are computed with the individual images 4. During the movement correction 7, the positions of the recorded individual images 4 are aligned. The video camera 1 or the object, i.e., the tissue or vascular area may move during video recording. In such cases, the individual images 4 are offset from each other. Thus, the individual images 4 must be re-aligned in order to evaluate the details visible in the individual image without faults. This is exacerbated by the constantly changing image information in the individual images. To have an initial image for comparison purposes, a reference image is selected from among the individual images. The first image on which clear structures can be recognized can serve as an initial reference image. Using an edge detection method, all individual images 4 that are to be computed with the reference image are continuously examined for their degree of offset in comparison to the reference image. This offset is taken into account in all additional steps where several individual images 4 are involved. In particular the reference image is continuously updated by integrating the edge image of the following individual image that is offset to the correct position into the reference image.

The brightness determination 8 can be carried out following the corrections 6 and 7. For this purpose, first the position of the measurement range is determined in a measurement range determination 11. The measurement range for which the superimposed representation has to be generated can be defined in a measurement range determination 11 via a measurement window or as a selection of specified measurement points. For example, a range of the recording can be selected if only this range is to have a superimposed representation, or if the quantities or the superimposition are to be generated for a portion of the image points only in order to save computing time. The result of the brightness determination 8 is a brightness plot 12 as a function of the time as can be seen in FIG. 2. This brightness plot 12 is computed for all or at least for a sufficiently large sample of image points.

In an evaluation 13, numerous other representations 14, comprising individual results as well, can be supplied from these brightness plots 12 and the individual images 4. They can then be represented on the screen together with the individual images 4.

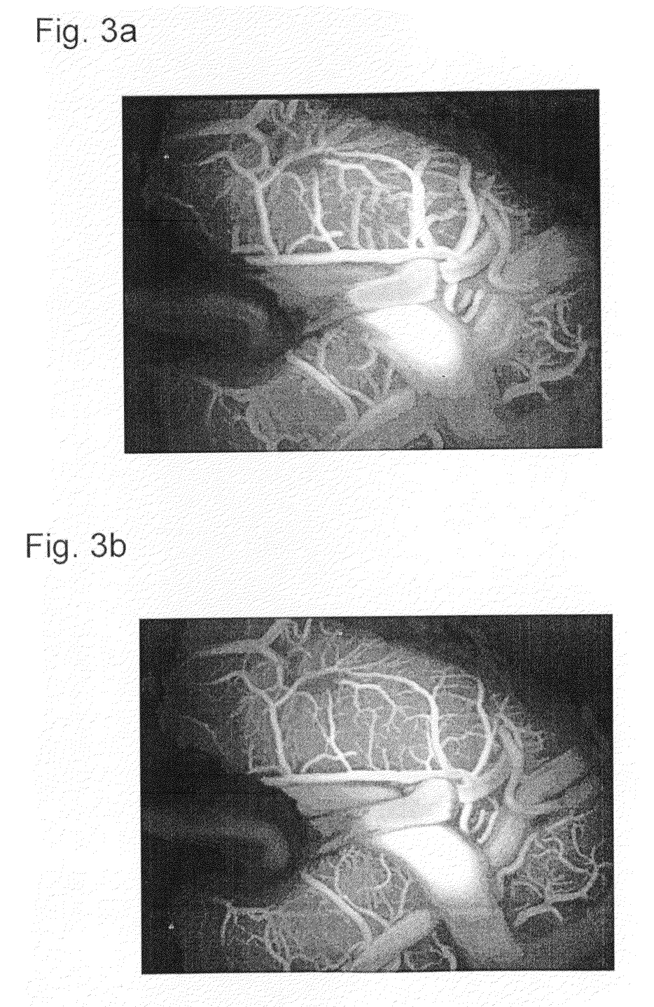

One example for this is a so-called blood vessel representation, where all vessels in which fluorescent agents have flowed and all tissues through which fluorescence agents flowed appear in white. This representation is generated by representing the difference between the maximum and minimum brightness value for each image point of the superimposed individual images 4. With this maximum brightness for each image point, one obtains a relative, quantitative quantity for the blood flow at all positions. This enables the physician to recognize defects. Examples for blood vessel representations can be seen in FIGS. 3a and 3b. FIG. 3a shows a blood vessel representation that has been generated without movement compensation 7, while FIG. 3b shows an example with movement compensation 7. Clearly recognizable is the significantly better sharpness of the contours in FIG. 3b with movement compensation.

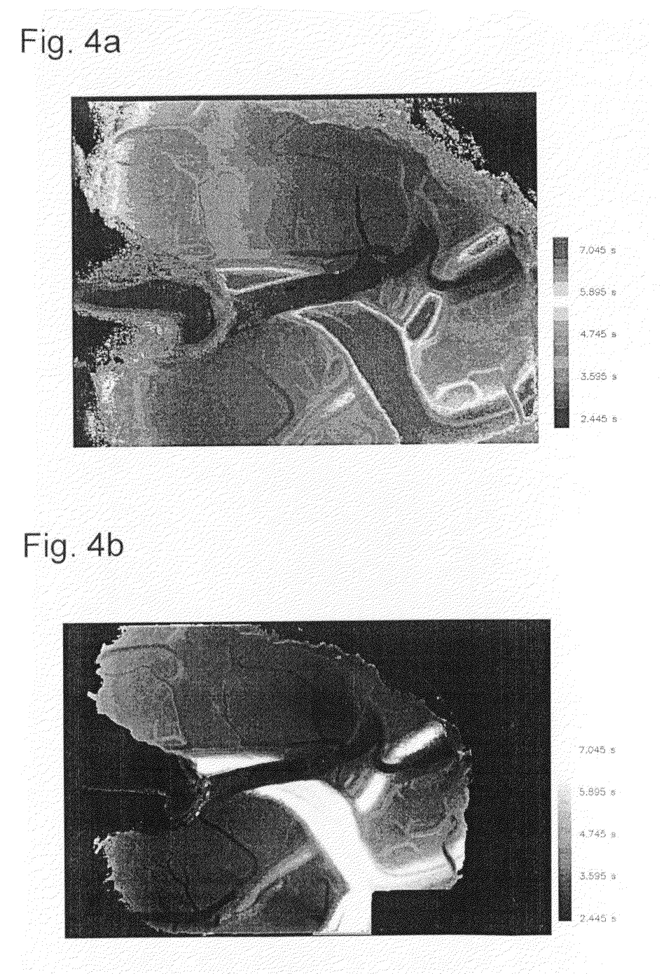

A two-dimensional false color image representing the time offset is provided for an additional representation 14. It can be seen in FIGS. 4a and 4b. FIG. 4a shows the onset time of the blood flow in a color representation transferred into grayscale, whereby the bars on the right side show the false color scale, the relationship between the selected colors and the respective relapsed time. The false color scale is selected such that an intuitive correlation to known anatomic terms exists. Accordingly, red is selected for an earlier point in time in order to emphasize the arterial character and blue for a later point in time to accent the venous character. In FIG. 4a, the color scale thus transitions from red (here at about 2.5 sec) to green (here at about 5 sec) and finally to blue (here at about 7 sec). In this manner, the physician receives a quick overview of the time when the blood arrived at which position of the blood vessel. Thus, using the time offset, information about the inflow and outflow of the blood in the blood vessels or in the tissue is made transparent. Because the transfer of the false color image into grayscale does not permit an unambiguous assignment of the colors, a similar representation 14 of a time offset in place of a false color image has been implemented as a grayscale image with a grayscale for black and white representations as are necessary here, for example, or also for black-and-white screens. This can be seen in FIG. 4b. Here, blood vessels into which the blood with the fluorescent dye flows immediately are shown dark while the blood vessels that the blood reaches later are shown very brightly. However, the grayscale representation has less information contents compared to the false color representation.

To generate the representation 14, a brightness plot 12 is computed for each image point based on all individual images 4 of the video. Then the point in time t₁ at which the brightness plot 12 has exceeded a certain threshold value I(t₁) is determined for each image point. The threshold value is defined as I(t₁)=Iₘᵢₙ+0.2×(Iₘₐₓ−Iₘᵢₙ). This point in time is converted to the respective color grayscale or height and entered into the time offset representation, Iₘₐₓ and Iₘᵢₙ must be determined by comparing the recorded data of several individual images 4 in order to determine the threshold value I(t₁). To obtain a spatially resolved signal, it is extremely important to carry out a movement compensation first. Without movement compensation 7, the brightness plot is not steady such that several Iₘₐₓ and Iₘᵢₙ could arise in each brightness plot 12. The same applies to the brightness correction 6. Without a brightness correction 6, a steady plot would also not arise for recording devices where the recording conditions may change during the recording of the individual images 4 and where the changes affect the brightness of the recorded individual images 4. Changes in the recording conditions may be necessary, for example, whenever a greater contrast range is to be covered.

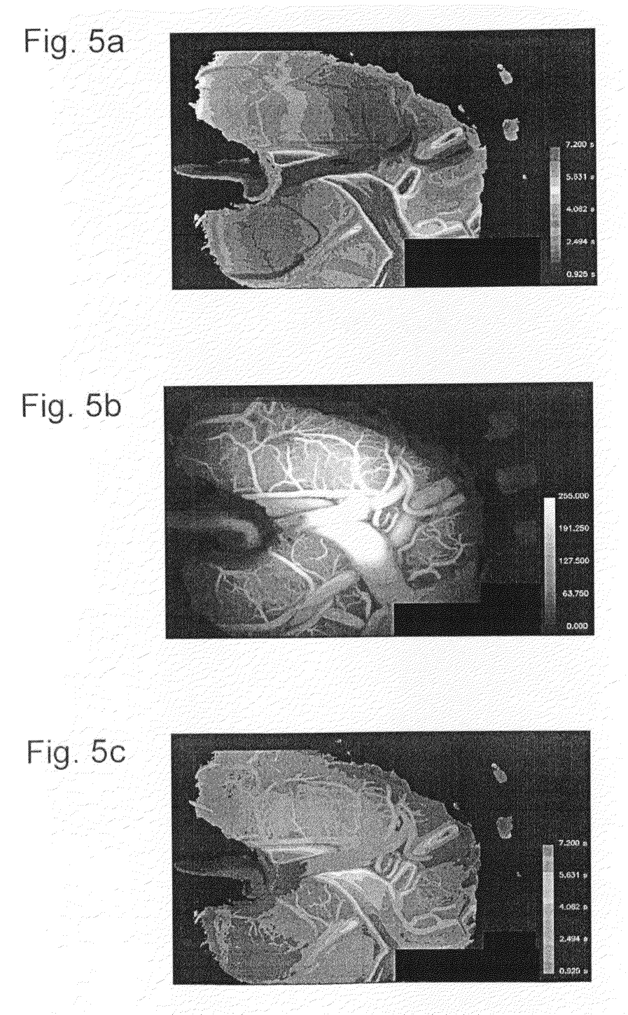

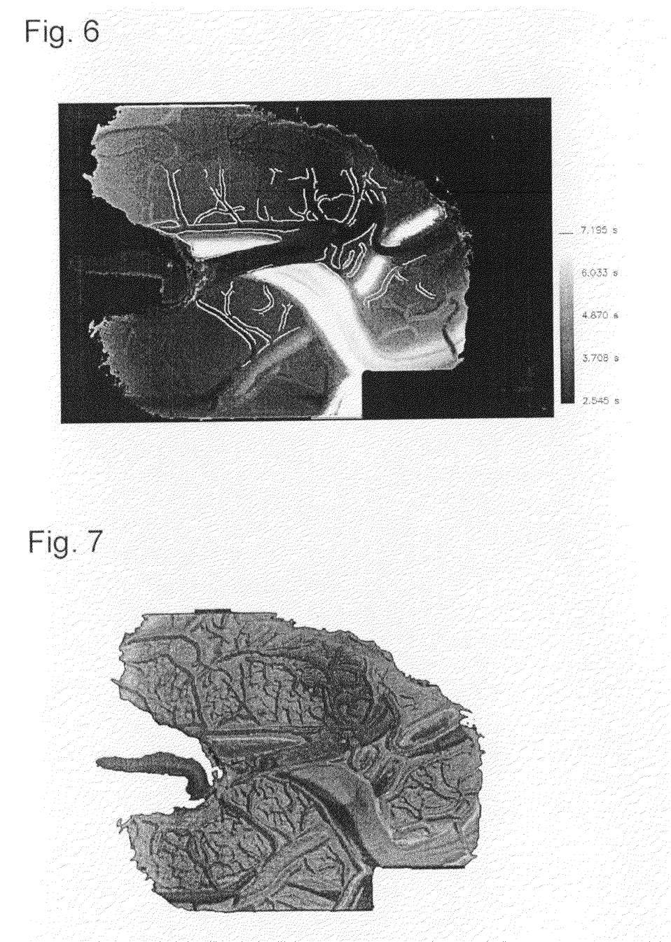

A problem in the qualitative and also quantitative representation of the blood flow, such as the time offset representation, for example, is that blood vessels or tissue regions that are close to each other often exhibit the same flow behavior and, therefore, melt into one region in the representation. In this manner, the flow behavior or the blood flow often can no longer be assigned unambiguously to one single vessel when viewing the representation. To re-enable this location assignment, a superimposition of a quantity that quantifies the blood flow at every image point and of a quantity that presents the position of the vessels is recommended in an additional representation as can be seen in FIGS. 5c, 6 and 7. FIG. 5c shows a superimposition of a time offset representation shown in FIG. 5a and explained based on FIG. 4 and a blood vessel representation shown in FIG. 5b and explained based on FIG. 3. The superimposition is weighted, i.e., one portion G1 each of the time offset is added to a portion G2 of the blood vessel representation, where the portions G1 and G2 build a sum of One. In the example of FIG. 5c, the superimposed representation is calculated as the superimposition=0.6×time offset representation+0.4×blood vessel representation. Although the representation shown in FIG. 5a again does not allow for a precise assignment of the inflow behavior, because the false color image has been converted to a grayscale image, it is apparent that in this representation layout entire regions exhibit the same flow behavior and, therefore, cannot be assigned to individual vessels. This is different in the superimposition shown in FIG. 5c. Here too the inflow behavior is represented by the color scale (here unfortunately converted to grayscale), however, it can be clearly localized due to the superimposed blood vessels and then can be assigned to the superimposed vessels. In this representation, both the moment in time of the inflow as well as the position of fine blood vessels is clearly recognizable. This can be seen even more clearly in FIG. 6. It shows a superimposition of a time offset representation as a grayscale image as described in FIG. 4b and of an edge image. The edge image is obtained from the blood vessel representation using an edge detection method. The superimposition is carried out by entering the value of the time offset at the image points where no edges appear, while entering a fixed value at image points where edges are present. The vessels appear more complete the lower the selected threshold value for the edges are. In the edge image that is superimposed in FIG. 6, the selected threshold is very high because the drawing shall only illustrate the principle. This representation provides a very clear overview over the path of the vessels and the blood flow that occurs in them.

One example for a superimposition in the form of a 3D representation can be seen in FIG. 7. This again is a superimposition of an already described quantity, where the time offset representation enters as a false color image and the blood vessel representation expands in the fashion of a relief as a third dimension into the space.

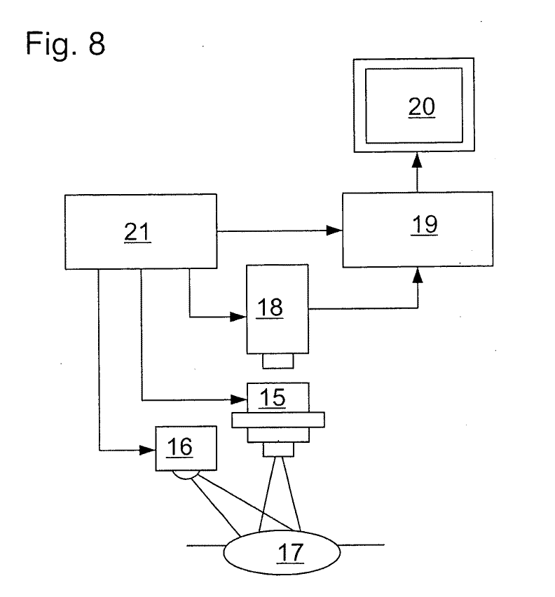

FIG. 8 shows schematically the essential components of a surgical microscope that can be used to apply the method according to the invention. The optics 15 of a surgical microscope reproduces an object 17, for example the head of a patient that is to be treated during surgery and is illuminated by a light source 16 in a camera 18. The camera 18 can also be a component of the surgical microscope. The image data recorded by the camera 18 are transferred to a computer unit 19 where they are evaluated. Medical quantities derived at the evaluation are then represented on the screen 20, potentially together with the recorded image. Similar to the computer unit 19, the screen 20 can be a component of the central surgical control but can also be a component of the surgical microscope. A control unit 21 controls the brightness of the light source 16 as well as the magnification factor and the aperture of the optics 15 and the amplification factor of the camera 18. In addition, the control unit 21 generates metadata that provide information about changes in the recording conditions that occur as soon as the control unit 21 adjusts a quantity that is to be controlled. These metadata are transferred from the control unit 21 to the computer unit 19, where they are assigned to the image data that have been provided to the computer unit 19 by the camera 18. Metadata and image data are stored, at least temporarily, by the computer unit 19 and are evaluated according to the method according to the invention. During the evaluation, the metadata are included with the image data. The results of the evaluation according to the invention are then displayed on the display unit 20, possibly together with the image data.

There has thus been shown and described a novel method and apparatus for quantitative display of blood flow which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

What is claimed is:

1. A method for the quantitative representation of the blood flow in a tissue or vascular region based on the signal of a contrast agent injected into the blood, said method comprising the steps of:
recording and storing, at successive points in time, several individual images of the signal emitted by the tissue or vascular region,
based on the respective signal, determining a quantity characteristic for the blood flow and a quantity characteristic for the position of the blood vessels for image areas of several individual images, and
representing these quantities superimposed for the respective image areas.

2. A method for the quantitative representation of the blood flow in a tissue or vascular region as set forth in claim 1, wherein the superimposition is carried out image point by image point.

3. A method for the quantitative representation of the blood flow in a tissue or vascular region as set forth in claim 1, wherein the superimposition is carried out via a weighted addition of quantities that are characteristic for the blood flow and for the position of the blood vessels.

4. A method for the quantitative representation of the blood flow in a tissue or vascular region as set forth in claim 1, wherein a multicolor representation is superimposed with a grayscale representation.

5. A method for the quantitative representation of the blood flow in a tissue or vascular region as set forth in claim 1, wherein the superimposition is carried out using a control quantity that is used to decide which quantity shall be employed for each image point of the superimposed representation.

6. A method for the quantitative representation of the blood flow in a tissue or vascular region as set forth in claim 5, wherein the control quantity is derived from the quantity that is characteristic for the position of the blood vessels.

7. A method for the quantitative representation of the blood flow in a tissue or vascular region as set forth in claim 5, wherein the quantity that is characteristic for the blood flow comprises a continuous data set and the quantity that is characteristic for the position of the blood vessels a binary data set.

8. A method for the quantitative representation of the blood flow in a tissue or vascular region as set forth in claim 7, wherein the quantity that is characteristic for the position of the blood vessel constitutes an edge image.

9. A method for the quantitative representation of the blood flow in a tissue or vascular region as set forth in claim 1, wherein a three-dimensional representation is selected for the superimposition of the quantities.

10. A method for the quantitative representation of the blood flow in a tissue or vascular region as set forth in claim 1, wherein the quantities to be superimposed are represented based on at least two quantities of the HSL color space (hue, saturation, luminance).

11. A method for the quantitative representation of the blood flow in a tissue or vascular region as set forth in claim 1, wherein the time offset is the quantity characteristic for the blood flow.

12. A method for the quantitative representation of the blood flow in a tissue or vascular region as set forth in claim 1, wherein the blood flow index is the quantity characteristic for the blood flow.

13. A method for the quantitative representation of the blood flow in a tissue or vascular region as set forth in claim 11, wherein the time offset or the blood flow index are represented in the form of a false color image.

14. A method for the quantitative representation of the blood flow in a tissue or vascular region as set forth in claim 13, wherein the superimposition is carried out using a grayscale image of the quantity that is characteristic for the position of the blood vessel.

15. A method for the quantitative representation of the blood flow in a tissue or vascular region as set forth in claim 11, wherein the time offset or the blood flow index are represented in the form of a grayscale image.

16. A method for the quantitative representation of the blood flow in a tissue or vascular region as set forth in claim 15, wherein the superimposition is carried out using an edge image of the quantity that is characteristic for the position of the blood vessels.

17. A method for the quantitative representation of the blood flow in a tissue or vascular region as set forth in claim 1, wherein the quantity that is characteristic for the position of the blood vessels is determined by comparing the intensity of different points in time for image areas of the individual images and in that the maximum intensities of the signal for this image area are determined.

18. A method for the quantitative representation of the blood flow in a tissue or vascular region as set forth in claim 1, wherein a movement compensation is applied for the individual images prior to the determination of the points in time.

19. A method for the quantitative representation of the blood flow in a tissue or vascular region as set forth in claim 18, wherein edge images of individual images are generated for the movement compensation using an edge detection method.

20. A method for the quantitative representation of the blood flow in a tissue or vascular region as set forth in claim 19, wherein edge images are correlated to each other in order to determine a shift factor.

21. A method for the quantitative representation of the blood flow in a tissue or vascular region as set forth in claim 20, wherein each correlation of the edge image of an individual image is carried out using a reference image that is developed by supplementing the edge images of two correlated and shifted individual images in the reference image.

22. A method for the quantitative representation of the blood flow in a tissue or vascular region as set forth in claim 21, wherein the developed reference image is used as a quantity that is characteristic for the position of the blood vessel.

23. A method for the quantitative representation of the blood flow in a tissue or vascular region as set forth in claim 1, wherein a brightness correction is applied for the individual images prior to the determination of characteristic quantities.

24. A method for the quantitative representation of the blood flow in a tissue or vascular region as set forth in claim 23, wherein metadata are recorded and stored for the brightness correction during recording of the individual images.

25. A surgical microscope for recording a fluorescence radiation of a contrast agent comprising a camera for recording a sequence of an object and optics for reproducing the object in the camera, wherein the camera is connected to a computer unit for deriving medical quantities from an image sequence of medical image data or individual images of the image sequence, the improvement wherein the computer unit operates in accordance with a program for carrying out the method as set forth in claim 1.

26. An analysis system, in particular a surgical microscope for recording a fluorescence radiation of a contrast agent, comprising a computer unit that operates in accordance with a program for performing the method as set forth in claim 1.
